(19) United States
(12) Patent Application Publication
(10) Pub. No.: US 2002/0055143 A1
(43) Pub. Date: May 9, 2002

(54) Bone precursor compositions

(75) Inventors: Eugene Bell, Boston, MA (US); Tracy M. Sioussat, Reading, MA (US)

Correspondence Address: Ellen Leonnig, TEI Biosciences, Inc., 7 Elkins Street, Boston, MA, 02127, US

(73) Assignee: Tissue Engineering, Inc.

(21) Appl. No.: 09/867,093

(22) Filed: May 29, 2001

Related U.S. Application Data
(63) Continuation of application No. 09/369,012, filed on Aug. 5, 1999.
(60) Provisional application No. 60/095,627, filed on Aug. 7, 1998.

(51) Int. Cl. C12P 21/02; A61K 45/00

(52) U.S. Cl. 435/69.1; 424/93.7

(57) ABSTRACT

Bone precursor compositions, methods of preparation and use are described. Bone precursor compositions include a calcium cement which is suitable for injection, wherein the calcium cement includes monobasic calcium phosphate monohydrate and beta-tricalcium phosphate. The bone precursor compositions can further include biopolymer foams, collagen, extracellular matrix components, therapeutic agents, or biopolymer fibers. The bone precursor compositions can also include or be conditioned with cells, such as connective tissue cells, preferably bone tissue cells.

RELATED APPLICATIONS

This application claims the benefit of priority under 35 U.S.C. 119(e) to copending U.S. Provisional Application No. 60/095,627, filed on Aug. 7, 1998, the entire contents of which are incorporated herein by reference.

### BACKGROUND OF THE INVENTION

Bone is a biological composite having a calcium phosphate mineral phase within a collagen biopolymer matrix phase. Bone has an average modulus of elasticity of about 20 GN/m², compressive strength of 170 to 220 MN/m², tensile strength of 180 MN/m² and bending strength of approximately 220 to 270 MN/m². As a composite, bone differs from other composite materials by possessing an orderly intimate combination of a calcium phosphate mineral phase within a collagen biopolymer matrix phase. Collagen is deposited by cells which organize the composite structure. The calcium phosphate appears to self-assemble at gaps in the collagen phase to create mineral-polymer composite fibers. These mineralized collagen fibers are bonded together in an orderly manner by further calcium phosphate cementation.

In many situations bone is broken, destroyed, degraded, or becomes too brittle. Alternatively, bone can be traumatized by various stressors, it can have naturally occurring gaps and/or defects. Various materials have been investigated which act as a support, substitute, or an interface for repairing or replacing naturally occurring bone structure. Bone replacement structures frequently do not bond to the affected bone, thereby providing a weak juncture which is subject to failure due to stresses associated with normal movement and use of the bone structure. For example, replacement materials, such as cobalt-chromium or titanium prostheses require that the interface between the bone and the prosthesis have a strong bond so that the prosthetic device is securely attached to the bone structure. To achieve this, a bone cement is generally used in conjunction with prosthetic implants.

The standard bone cement currently used in orthopedic surgery is poly(methylmethacrylate) (PMMA). A common complication associated with implants cemented with PMMA is that the implant loosens over time due to everyday stresses placed upon the implant/cement/bone interface. Further complications can be associated with the breakdown of PMMA as a result of mechanical fatigue and subsequent degradation in the physiological environment. Additionally, when PMMA is used to fill large bone areas, the heat of polymerization often results in temperatures high enough to cause deep necrosis of the surrounding bone tissue. Additionally, the initial toxicity of the methyl methacrylate monomer and the non-resorbability of PMMA preclude its use for bone grafting.

Cement-like biomaterials offer considerable advantages over these standard bone cements since they can be shaped and hardened in situ, thereby affording the best possible fit with the surrounding bone tissue. Various calcium phosphate formulations have been proposed as resolvable biomaterials. These formulations typically consist of aqueous mixtures of calcium phosphates, such as monocalcium phosphate monohydrate, dicalcium phosphate anhydrous, dicalcium phosphate dihydrate, octacalcium phosphate, alpha-tricalcium phosphate, beta-tricalcium phosphate, tetra-calcium phosphate monoxide and calcium carbonate. The feature common to these formulations is that they combine a relatively basic calcium phosphate with a more acidic material thereby forming a phosphate of intermediate acidity. However, the compositions of these cements results in cements with several deficiencies which limit their practical use. For example, some cements set very rapidly (in less than 30 seconds), making it difficult or impossible for a surgeon to inject it into or at the desired location. In contrast, some cements set too slowly. In addition, the final diametral strength of some of these cements is rather low (less than 1 MPa) and decreases upon prolonged aging at physiological conditions.

### SUMMARY OF THE INVENTION

The present invention provides new cement formulations which are injectable, have setting times which enable their manipulation in vivo and which maintain their strength in physiological environments.

The invention is based, at least in part, on the discovery that bone precursor compositions of the invention can be prepared with the advantageous properties of being injectable, have set times which are between about 1 to about 15 minutes, preferably between about 5 to about 10 minutes, and/or are biodegradable and biocompatible and have high diametral strength which does not decrease upon aging at physiological conditions. The bone precursor compositions of the invention can be further modified with cells, reinforcing materials, with pore generating materials, extracellular particulates or fibrillar collagen, for example, to further improve the compatibility of the cement with the surrounding tissue into or onto which it is injected.

Accordingly, the invention pertains to a bone precursor composition including a calcium cement which is suitable for injection, wherein the calcium cement includes monobasic calcium phosphate monohydrate and beta-tricalcium phosphate. In a preferred embodiment, the bone precursor composition further includes calcium pyrophosphate and alpha-calcium sulfate hemihydrate wherein the ratio by weight of monobasic calcium phosphate monohydrate to beta-tricalcium phosphate is between about 1:2 to about 1:3.75, more preferably about 1:3.5 and most preferably about 1:3.05. In one embodiment the reacted and hardened calcium cement is in the form of granules with a diameter of between about 1 to 500 μm, preferably 50 to about 500 μm inclusive, preferably between about 100 to about 400 μm inclusive, most preferably between about 250 μm and about 350 μm inclusive. These granules can be formed by mechanical action such as grinding and sifting or sorting by size. In a preferred embodiment, fibrillar collagen is included in the bone precursor composition. In another preferred embodiment, the composition comprises unassembled liquid collagen.

Advantageously, the bone precursor compositions of the invention are injectable and have selected setting times and compression strengths which render them suitable for use as bone precursor compositions. In a preferred embodiment, the bone precursor composition includes or is conditioned with cells, such as those described infra. Bone precursor compositions of the invention can further include therapeutic agents or biopolymer fibers, e.g., collagen, such as porcine collagen.

The bone precursor compositions of the invention can include cells or can be conditioned with cells prior for use in vitro or in vivo, for example, to render the compositions suitable for use in vivo as prosthetic implants, or injectable compositions for replacement of damaged or diseased bone or to provide scaffolds which, when occupied by cells, e.g., host cells, are remodeled to become functional tissue such as bone. These compositions can be used for in vitro development of bone, to be implanted as a complete living replacement. This development may require mechanical or electrical conditioning to stimulate strengthening and tissue organization of the product to authentic magnitudes. Optionally, these bone precursor compositions can be used as model systems for research. In either case, the bone precursor compositions and constructs can be seeded with cells, e.g., mammalian cells, e.g., human cells, of the same type as those of that tissue which the bone precursor composition or connective tissue is used to repair or reconstruct. Examples of cells which can seeded onto the bone precursor compositions and constructs described herein include tissue cells or mesenchymal cells such as connective tissue or bone cells. Suitable examples of soft connective tissue cells include ligament cells, tendon cells and chondrocytes. Suitable examples of bone cells include bone marrow stem cells, osteocytes, osteoblasts and osteoclasts. In one embodiment, the bone precursor compositions and constructs seeded with tissue specific cells are introduced into a recipient, e.g., a mammal, such as a human. Typically, the cells included in the bone precursor compositions, or the cells which are used to condition the bone precursor compositions, are connective tissue cells such as mammalian connective tissue cells, e.g., fibroblastic ligament cells and chondrocytes, and/or bone cells such as bone marrow stem cells, osteocytes, osteoblasts and osteoclasts.

In another aspect, the invention pertains to bone precursor compositions which include calcium cement and a biopolymer structure, e.g., a foam or matt. The biopolymer foam can be a single density biopolymer foam or a double density biopolymer foam. In a preferred embodiment, either or both the calcium cement and the biopolymer foam or matt includes or is conditioned with cells.

In yet another aspect, the invention pertains to bone precursor compositions which include a calcium cement and acid or pepsin extracted collagen. The acid or pepsin extracted collagen can be in the form of lyophilized collagen or microfibrillar collagen, e.g., microfibrillar collagen in the form of a semisolid pellet. In a preferred embodiment, the collagen in the bone precursor compositions is between about 0.1 to 2.5 dry weight percent of the composition.

In still another aspect, the invention pertains to bone precursor compositions which include a calcium cement and macromolecules necessary for cell growth, morphogenesis, differentiation and tissue building, particularly in the form of extracellular matrix particulates. The extracellular matrix particulates can be between about 0.05 to 20 weight percent of the composition and the ratio by weight of monobasic calcium phosphate monohydrate to beta-tricalcium phosphate is between about 1:2 to between about 1:3.75, more preferably between about 1:3.5 and most preferably about 1:3.05. Alternatively, the reacted hardened calcium cement is in the form of granules with a diameter of between about 1 to 500 μm, preferably 50 to about 500 μm inclusive, preferably between about 100 to about 400 μm, most preferably between about 250 μm to about 350 μm. Additionally, the compositions can be conditioned with cells and/or growth differentiation or morphogenesis factors.

In a still further aspect, the invention pertains to a method for preparing a bone precursor composition suitable for injection. This method includes combining calcium pyrophosphate, alpha-calcium sulfate hemihydrate, monobasic calcium phosphate monohydrate and beta-tricalcium phosphate such that a bone precursor composition is prepared. In a preferred embodiment, the bone precursor composition includes calcium pyrophosphate and alpha-calcium sulfate hemihydrate wherein the ratio by weight of monobasic calcium phosphate monohydrate to beta-tricalcium phosphate is about 1:2 to about 1:3, preferably about 1:3.5, more preferably about 1:3.75 and most preferably about 1:3.05. In one embodiment the composition is in the form of granules with a diameter of between about 1 to 500 μm, preferably 50 to about 500 μm inclusive, preferably between about 100 to about 400 μm, most preferably between about 250 μm to about 350 μm. In a particularly preferred embodiment, fibrillar collagen is included in the bone precursor composition.

In a preferred embodiment, the method further includes the step of contacting, e.g., immersing or soaking, the reacted, hardened bone precursor composition with a neutralizing solution such that a neutralized bone precursor composition is prepared. The neutralizing solution is selected from the group consisting of CAPS, triethanolamine, TES, tricine, HEPES, glycine, phosphate buffer solution, bis tris propane, TAPS, AMP and TRIS, preferably tribasic sodium phosphate. The bone precursor composition can then be implanted or can be seeded with cells.

In still yet another aspect, the invention pertains to methods for producing or repairing connective tissue in a subject, comprising administering a bone precursor composition to the subject, wherein the bone precursor composition includes calcium pyrophosphate, calcium sulfate hemihydrate, monobasic calcium phosphate monohydrate and beta-tricalcium phosphate and, optionally, fibrillar collagen.

In addition, the injectable bone precursor compositions of the invention can include pharmaceutically acceptable injection vehicles, such as methylcellulose, saline, etc. Examples of other suitable injection vehicles include microfibrillar collagen or calcium cement, e.g., injectable calcium cement which includes calcium salts such as calcium pyrophosphate, alpha calcium sulfate hemihydrate, monobasic calcium phosphate monohydrate and beta tricalcium phosphate and fibrillar collagen. When injectable calcium cement is used as a vehicle it typically comprises, by weight, between about 1 and 5 percent, preferably about 1 percent, calcium pyrophosphate, between about 5 and 15 percent, preferably about 10 percent, alpha-calcium sulfate hemihydrate, between about 5 and 25 percent, preferably about 22 percent, monobasic calcium phosphate monohydrate and between about 55 and 75 percent, preferably about 67 percent, beta-tricalcium phosphate. In one embodiment, the calcium cement further includes semisolid microfibrillar collagen in an amount of about 20-50% additional wet weight, more preferably about 30-50%, and in another embodiment, at least about 35%.

The bone precursor compositions and constructs, with or without in vitro development, with or without cells or extracellular matrix particulates can be used, for example, as orthopedic implants, maxillofacial implants, dental implants, connective tissue implants, e.g., cartilage implants, bone replacement implants. Particularly, the bone precursor compositions and constructs which are used as orthopedic and/or dental implants include a calcium cement, e.g., a mixture of monobasic calcium phosphate monohydrate and beta-tricalcium phosphate, and optionally, calcium sulfate, calcium pyrophosphate or collagen. An example of such an implant is an alveolar ridge builder or a bone void filler pellet.

### DETAILED DESCRIPTION OF THE INVENTION

The present invention pertains to bone precursor compositions which include a calcium cement which is suitable for injection. The injectable calcium cement includes monobasic calcium phosphate monohydrate and beta-tricalcium phosphate. One embodiment further includes calcium pyrophosphate and alpha-calcium sulfate hemihydrate, preferably the ratio of dry weight of monobasic calcium phosphate monohydrate to beta-tricalcium phosphate is between about 1:2 to about 1:3.75, more preferably about 1:3.5 and most preferably about 1:3.05. In a preferred embodiment, the reacted and hardened calcium cement is in the form of granules with a diameter of between about 1 to 500 μm, preferably 50 to about 500 μm inclusive, preferably between about 100 to about 400 μm, most preferably between about 250 μm to about 350 μm. In a particularly preferred embodiment, collagen, e.g., fibrillar collagen, is included in the bone precursor composition. In an even more preferred embodiment, the bone precursor composition as either the mixture or in reacted and hardened form can include or can be conditioned for cell growth, conditioned with cells, or treated with macromolecules necessary for cell growth, morphogenesis, differentiation and tissue building, particularly in the form of extracellular matrix particulates. The bone precursor composition to be conditioned can be in the form of hardened pellets or a unitary structure formed before implantation, e.g., an implant.

The language “bone precursor composition” is intended to include those materials, such as the calcium cement compositions described herein, which can be used to form, repair, or replace damaged connective tissue, e.g., such as bone tissue In a preferred embodiment, the bone precursor composition is bioabsorbable and biocompatible. Preferably the base precursor composition is suitable for injection.

“Bioabsorbable,” as that term is used herein, means materials which are degraded in response to contact with body fluid or cells while implanted or injected in vivo. Examples of bioabsorption processes include hydrolysis, enzymatic action, oxidation and reduction. Suitable conditions for hydrolysis, for example, include exposure of the bioabsorbable materials, e.g., calcium cements of the invention, to water at a temperature and a pH of body fluids. Bioabsorption of cements of the present invention can be enhanced in low pH regions of the mammalian body, e.g. an inflamed area. Additionally, the cements of the invention will be remodeled by host cells over time and will disappear as it is replaced by new bone.

“Biocompatible,” as that term is used herein, means exhibition of essentially no cytotoxicity while in contact with body fluids. Both the material and its degradation products are nontoxic. “Biocompatibility” also includes essentially no adverse interactions with recognition proteins, e.g., naturally occurring antibodies, cell proteins, cells and other components of biological systems. However, substances and functional groups specifically intended to cause the above effects, e.g., drugs and prodrugs which may be added, are required to be biocompatible. The biocompatible cement compositions of the invention will not cause adverse tissue reactions such as an immune rejection or persistent inflammatory or foreign body response.

The term “calcium cement” is art recognized and is intended to include a material which when combined with a liquid initiator undergoes a chemical reaction and/or a crystal rearrangement which results in a cured, e.g., hard, solid. Via this setting reaction, the calcium cement can be used as a joiner, or filler for the assembling of connective tissue surfaces e.g., bone tissue, which are not in direct contact, and to bond bone tissue to metallic or synthetic prosthetic devices. Calcium cements can include an initiator for the setting reaction. A physiologically acceptable aqueous initiator, e.g., water or an aqueous buffer, can be used, such as aqueous solution, which can further include additional ingredients such as methylcellulose or collagen, e.g., microfibrillar collagen. The water which is used will be substantially pure, such as double-distilled or deionized or an equivalent thereof. Other hydroxyl containing materials e.g., methylcellulose, which are water miscible, pharmacologically acceptable and do not interfere with bone precursor formation, also find value as lubricants or injection vehicles.

The language “suitable for injection” is intended to include those bone precursor compositions and calcium cements which have physical characteristics which render the materials suitable for passage as a homogenous paste through a syringe needle e.g., typically a 14-22 gauge needle without clogging the needle or separating into liquid and solid phases.

The term “monobasic calcium phosphate monohydrate” is art recognized and is intended to include the compound defined as CaH₄(PO₄)₂/H₂O and has a calcium to phosphorous ratio of 0.5.

The term “beta-tricalcium phosphate” is art recognized and is intended to include the compound having the chemical formula of Ca₃(PO₄)₂ and has a calcium to phosphorous ratio of 1.5.

The term “calcium pyrophosphate” is art recognized and is represented by the formula Ca₂P₂O₇ and has a calcium to phosphorous ratio of 1.

The term “alpha-calcium sulfate hemihydrate” is art recognized and is represented by the formula CaSO₄ 0.5/H₂O.

The language “includes or is conditioned with cells” is intended to include bone precursor compositions which have cells attached or adhered to the calcium cement and can attach and grow fqr a sufficient period of time for deposition of informational macromolecules onto the cement. For example, cells contemplated by the invention include tissue cells or mesenchymal cells which include connective tissue cells or bone cells. Connective tissue cells further include ligament cells, tendon cells and chondrocytes. Bone cells are selected from bone marrow stem cells, osteocytes, osteoblasts and osteoclasts.

The term “mesenchymal cell” is art recognized and is intended to include undifferentiated cells found in mesenchymal tissue, e.g., undifferentiated tissue composed of branching cells embedded in a fluid matrix which is responsible for the production of connective tissue, blood vessels, blood, lymphatic system and differentiates into various specialized connective tissues.

The term “connective tissue” is art recognized and is intended to include primary tissue, which is distinguished by an abundance of fibrillar and non-fibrillar extracellular components and cells organized to support or surround other specialized tissue.

The term “bone cells” is art recognized and is intended to include osteoblasts, osteoclasts and osteocytes.

The term “fibroblast” is art recognized and is intended to include cells found in connective tissues.

The term “tendon cell” is art recognized and is intended to include those cells which when organized into a tendon connect a muscle to bone and permit concentration of muscle force into a small area.

The term “chondrocytes” is art recognized and is intended to include cartilage cells.

The term “bone marrow stem cells” is art recognized and is intended to include cells which can differentiate into mature blood and lymphatic cells or cartilage or bone cells.

The term “osteocytes” is art recognized and is intended to include those cells found within the lacunae, which are osteoblasts that have matured and have become incorporated within the bone matrix.

The term “osteoblasts” is art recognized and is intended to include those cells found most abundantly along bone-forming surfaces and have receptors for parathyroid hormone and are involved with the synthesis of osteocalcin, collagen I, alkaline phosphatase, osteonectin and assist in bone mineralization.

The term “osteoclasts” is art recognized and is intended to include monocyte-macrophage cells which are multinucleated cells found along the cortical endosteal surface and the trabeculae in scalloped bays (Howship's lacunae) where mineralized bone is being actively resorbed. These cells contain tartrate-resistant acid phosphatase, collagenases, dehydrogenases, proteases, and carbonic anhydrase. Signals from osteoblasts appear to be involved in activation of osteoclastic bone resorption.

The bone precursor composition can be pre-cast into a form, e.g., an implant, or pellets, e.g., particles, or a calcium cement which is suitable for injection. The injectable composition can further include a pharmaceutically acceptable vehicle, or preferably, microfibrillar collagen. The injectable composition noninvasively fills voids and hardens there as a resilient bone replacement or prevents the motion of small bone fragments during healing. The pellets can be placed and contained in open voids to augment the repair of large or irregular defects.

The phrase “pharmaceutically acceptable vehicle” is art recognized and includes a pharmaceutically acceptable material, composition or carrier, suitable for administering bone precursor compositions of the invention to mammals by injection. The vehicles include liquid or solid filler, diluent, excipient, solvent or encapsulating material, involved in carrying or transporting the bone precursor composition from a syringe to the cavity in need thereof. Each carrier must be “acceptable” in the sense of being compatible with the other ingredients of the formulation and not injurious to the patient. Some examples of materials which can serve as pharmaceutically acceptable vehicles, include: sugars, such as lactose, glucose and sucrose; starches such as cornstarch and potato starch; cellulose and its derivatives, such as sodium carboxy methylcellulose, ethyl cellulose and cellulose acetate; powdered tragacanth; malt; gelatin; talc; excipients, such as cocoa butter and suppository waxes; oils such as peanut oil, cottonseed oil, safflower oil, sesame oil, olive oil, corn oil and soybean oil; glycol such as propylene glycol; polyols such as glycerin, sorbitol, manitol and polyethylene glycol; esters, such as ethyl oleate and ethyl laurate; buffering agents such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants such as sodium lauryl sulfate and magnesium stearate, as well as coloring agents, stabilizers, preservatives or antioxidants can also be present in the compositions.

Methods of preparing these formulations or compositions include the step of bringing into association the calcium salts of the present invention with an initiator which can include a carrier and, optionally, one or more accessory ingredients. In general, the formulations are prepared by uniformly and intimately bringing into association the calcium salts of the present invention with liquid initiators which can include carriers or finely divided solid additives, or both, and then, if necessary, shaping the product.

In one embodiment, the bone precursor composition further includes solid additives (“pore-generating particles”) which bioabsorb more quickly than the calcium salts of the composition, thereby causing the bone precursor composition to become porous. For example, bioabsorbable particles having a diameter of between about 20 to about 250 μm inclusive can be added to the bone precursor compositions. Suitable bioabsorbable particles or pore-generating particles include gelatin, hardened calcium sulfate, salt or sugars, generally in a 5 to 70% range by dry weight to bone precursor composition. Porous bone precursor compositions provide the advantage of being suitable for osteoconduction.

Liquid dosage forms suitable for administration of the bone precursor compositions of the invention include pharmaceutically acceptable emulsions and microemulsions, solutions, suspensions, syrups and elixirs. In addition to the active ingredients, e.g. calcium salts, the liquid dosage form can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propyleneglycol, 1,3-butyleneglycol, oils (in particular, cottonseed, groundnut, corn, germ, olive, castor and sesame oils), glycerol, tetrahydrofuryl alcohol, polyethyleneglycols and fatty acid esters, sorbitan and mixtures thereof.

The bone precursor compositions can also contain adjuvants such as preservatives, wetting agents, emulsifying agents and dispersing agents. Prevention of the action of microorganisms may be insured by the inclusion of various anti-bacterial and anti-fungal agents, for example, paraben, chlorobutanol, phenol sorbic acid, and the like. It may also be desirable to include isotonic agents, sugars, sodium chloride and the like into the compositions. In addition, prolonged absorption of the injectable bone precursor compositions can be brought about by the inclusion of agents which allay absorption such as aluminum monostearate and gelatin e.g., collagen.

A preferred vehicle is microfibrillar collagen. The collagen used in the compositions and foams of the invention can be in the form of collagen microfibrils. In another preferred embodiment, the vehicle is unassembled liquid collagen.

The language “collagen microfibril” is art recognized and is intended to include collagen in the form described in Williams, B. R. et al. (1978) J. Biol. Chem. 253 (18):6578-6585 and U.S. patent application Ser. No. 08/910,853, filed Aug. 13, 1997, entitled “Compositions, Devices, and Methods for Coagulating Blood” by Eugene Bell and Tracy M. Sioussat, the contents of which are incorporated herein by reference. In a preferred embodiment, the collagen microfibrils are prepared as a semisolid (viscous) pellet of collagen microfibrils resulting from centrifugation of a neutralized solution of collagen. For example, the collagen can be neutralized by liquid 0.01-2.0 N NaOH, 0.1-10% ammonium hydroxide, or other known neutralizing solutions, before spinning in a centrifuge to yield a microfibrillar collagen pellet mass. The liquid content of the microfibrillar collagen pellet mass can be manipulated by the relative centrifugal force employed. For example, the stronger the centrifugal force, the less liquid and the higher the resulting concentration of microfibrillar collagen (e.g., from about 10 to about 100 mg/ml). The resultant semisolid pellet of neutralized microfibrillar collagen can be manipulated like a fluid such that it can be propelled from, for example, a device of the present invention, onto or into a site of bleeding. Since the microfibrillar collagen is already neutral, no gelling is required. However, the density of this form of collagen allows it to remain in place at the desired site of bleeding. The structure of the microfibrillar collagen provides the surface to initiate the clotting cascade at the site of bleeding.

Methods for purifying collagen so it can form microfibrils typically include the steps of extracting proteins from, for example, the skin of an animal, e.g., chicken, mammal, e.g., a marine mammal, a cow, goat, sheep, or, preferably, a pig, e.g., a fetal or newborn pig. This extraction step involves the use of organic acid such as formic or acetic acid. The collagen is then precipitated from the extract by salt (e.g., sodium chloride up to 3.0M or ammonium sulfate up to 50%) and collected by centrifugation. The collagen is then redissolved in organic acid and concentrated. The collagen can then be used or subjected to as many rounds, e.g., two rounds, of salt precipitation and centrifugation as desired before concentrating and using in the present invention. A preferred collagen concentration used to make microfibrillar collagen is 4.0 to 10.0 mg/ml. An alternative method for purifying collagen includes a method in which pepsin is included in the extraction acid solution, with all other steps the same as described above, with the additional updated steps described below.

For further details on the methods for purifying collagen, see U.S. Pat. No. 5,562,946 (hereinafter the “'946 patent”), the corresponding PCT application of which was published on May 17, 1996 and assigned International Publication No. WO 96/14452, the contents of both of which are incorporated herein by reference. This purification method is described at columns 6-8 of the '946 patent has been updated as follows: at lines 57-61, of column 7, rather than dialysis bags for dialysis, hollow fiber membranes are used with a 0.1 μm cutoff (or 100,000 MW for pepsin collagen). Thus, the centrifugation step at lines 62-64 of column 7, occurs before the dialysis step and the concentration step described at 66-67 of column 7 and lines 1-4 of column 8 occurs at the same time as the dialysis in the same hollow fiber.

In certain embodiments of the invention, calcium cement in the form of granules can be admixed with an injection vehicle which includes microfibrillar collagen, unassembled liquid collagen (e.g., at a concentration of about 5 mg/ml to about 40 mg/ml) or a calcium cement of calcium pyrophosphate, alpha-calcium sulfate hemihydrate, monobasic calcium phosphate monohydrate, beta-tri-calcium phosphate and, optionally, a wetting agent. In one embodiment, the mixture can be conditioned with cells. These particles can be injected into places where they can disperse and infiltrate into multiple small cavities to initiate bone regrowth throughout the interior of bones weakened by osteoporosis.

The addition of unassembled liquid collagen to the injection vehicle gives the cement the capability of forming a semi-solid unit with the cement particles trapped in a collagen gel. While the unassembled collagen is a liquid in the pH 3 to 6 suspension prior to injection, after injection and contact with neighboring neutral pH tissue of the patient, the collagen can assemble into a gel of sufficient structure to hold the particles in their three dimensional suspended positions. This injectable composition, which results in a gelled suspension of the cement particulates is useful as a highly osteoconductive material for filling larger interior voids than practical with microfibrillar collagen injection vehicle.

A most preferred embodiment of cement includes by dry weight, 1% calcium pyrophosphate, 10% alpha-calcium sulfate hemihydrate, 22% monobasic calcium phosphate monohydrate and 67% beta-tricalcium phosphate and, optionally, between about 0.1 to about 2.5% collagen by dry weight.

The use of the injectable cement injection vehicle does provide the microparticulates with a structural binder. The cement binder and the collagen binder have some important differences, such as the degree of porosity, the degree of structural strength and the rate of remodeling. The cement binder will result in a structure of higher strength, lower porosity, and slower remodeling rate than the gel binder. A person of skill in the art will be able to apply expertise in deciding the appropriate binder for the situation. Self assembling molecules, such as fibrinogen and certain synthetic polymer precursors are also suitable agents for inclusion in the injection vehicle for the purpose of binding the cement particles in suspension after injection. However, fibrinogen already is present at the injection site in the blood supply and, through natural clotting mechanisms, may form a fibrin gel clot in conjunction with or adjacent to the collagen gel or microfibrils in any injected composition of cement particles. Synthetic polymer precursors form materials of less instructive value than collagen and are not actively remodeled by host tissue while they degrade by hydrolytic mechanisms. Cells and liquids can transverse the collagen gel and cells can bind to it. Bound cells can remodel the collagen into structures they need or they can associate into tissues using the collagen filament framework and rebuild the bone at the injected site, with the cement microparticulates giving a jump-start of calcification. In a further embodiment, the injection vehicle may also, advantageously, include materials which increase the viscosity of the composition such as, for example, microfibrillar collagen, collagen foam, collagen fiber particles, or 0.1 to 15%, more preferably 0.5 to 10%, methyl cellulose. The injection vehicle may also comprise a pharmaceutically acceptable carrier as mentioned supra.

Bone precursor compositions can include therapeutic agents. For example, therapeutic agents include antibiotics, such as gentamycin, penicillin, streptomycin, anti inflammatory agents, such as cyclosporin, and/or agents such as cytokines, growth factors, or macromolecules necessary for growth, morphogenesis, differentiation, or tissue building, or extracellular matrix particulates.

As indicated above, a bone precursor composition of the invention can be fabricated with biopolymer fibers. For example, a biopolymer fiber, a multi-fiber element, or a biopolymer fabric comprising fibers can be embedded in or about cement. The cement can serve as an anchor for fibers embedded in the cement, for example, in a ligament replacement where the cement anchors the ligament precursor fibers in the bone at the site of ligament attachment Alternatively, the calcium cement can be deposited into these fibers in the form of a coating or in granulated form. Methods and apparatus for fabricating biopolymer fibers are known to those with ordinary skill in the art as disclosed in U.S. Pat. No. 5,562,946, entitled “Apparatus and Method for Spinning and Processing Collagen Fiber,” issued Oct. 8, 1996 and herein incorporated by reference. Preferably, the biopolymer fiber is formed of collagen, most preferably from fetal porcine collagen.

The term “biopolymer” as used herein, is intended to include naturally occurring polymers or man-made polymers from naturally-occurring components which are suitable for introduction into a living organ, e.g. a mammal, e.g., a human. Preferably, the biopolymer is non-toxic and bioabsorbable when introduced into a living organism and any degradation products of the biopolymer are also non-toxic to the organism. Biopolymers of the invention can be formed into structures such as biocompatible foams, e.g. single or double density foams, composite foams, and biocompatible constructs within or attached to bone precursor composition which include biopolymer fibers, e.g., collagen fibers, biopolymer fabrics, e.g., collagen fabrics, and/or extracellular matrix particulates. Examples of molecules which can form biopolymers which can be used in the present invention include collagen, thrombospondin, gelatin, polysaccharides, poly-l-amino acids, elastin, laminin, heparin sulfate proteoglycan, fibronectin and fibrinogen and combinations thereof. For example, a combination of collagen with a calcium cement can form a bone precursor composition.

Preferred sources of molecules which form biopolymers include mammals such as pigs, e.g., near-term fetal pigs, sheep, and cows. Other sources of molecules which can form the biopolymers include both land and marine vertebrates and invertebrates. In one embodiment, the collagen can be obtained from skins of near-term, domestic porcine fetuses which are harvested intact, enclosed in their amniotic membranes. Collagen or combinations of collagen types can be used in the foams, fibers, and foam compositions described herein. Examples of collagen or combinations of collagen types include collagen type I, collagen type II, collagen type III, collagen type IV, collagen type V, collagen type VI, collagen type VII, collagen type VIII, collagen type IX, collagen type X, collagen type XI, collagen type XII, collagen type XIII, and collagen type XIV. A preferred combination of collagen types includes collagen type I, collagen type III, and collagen type IV. Preferred mammalian tissues from which to extract the molecules which can form biopolymer include entire mammalian fetuses, e.g., porcine fetuses, dermis, tendon, muscle and connective tissue. As a source of collagen, fetal tissues are advantageous because the collagen in the fetal tissues is not as heavily cross linked as in adult tissues. Thus, when the collagen is extracted using acid extraction, a greater percentage of intact collagen molecules is obtained from fetal tissues in comparison to adult tissues. Fetal tissues also include various molecular factors which are present in normal tissue at different stages of animal development.

The biopolymers can be used to create sponges or foams, e.g., single or double density foams, which can be in any form or shape, e.g., strips, sheets, tubes, etc. In addition, the biopolymers can be used to create foams which are then combined with cement composition to form implants, such as a cement overlaid with a single density foam to produce an osteochondral replacement to repair an articular joint.

The forms and shapes in which the foams and foam compositions are made can mimic those of tissues or body parts to be replaced and thus can be used as prostheses or grafts which tissue cells remodel to promote regeneration of a replacement tissue in the recipient. Single density or double density foam compositions which are useful with the cement compositions of the invention are described in U.S. patent application Ser. No. 08/754,818, filed Nov. 21, 1996, entitled “Biopolymer Foams for Use in Tissue Repair and Reconstruction” by Bell et al., now U.S. Pat. No. 5,891,558, the contents of which are expressly incorporated herein by reference. Extracellular matrix particulates and/or viable cells can also be added to the biopolymers to further promote cell in growth and tissue development and organization within the foams.

For example, single density foams can be cast into the inner portion of a preformed cement tube. This composite can replace bone segments, with the cement providing the replaceable structural bone cortex and the foam core allowing the regrowth of the bone marrow. Alternatively, a single or double density foam can be cast onto a hardened cement form, or preferably adding a foam to a cement composition which is still hardening and includes collagen within the cement form. Reacted, hardened cement particulates can be mixed with collagen and cast into a foam with the particles suspended in the foam matrix. Particle density ranges from 0.5 to 40% particle weight to wet collagen volume. The foam and/or the cement can be further treated with extracellular matrix particulates and/or viable cells as described above. This material provides an immediately osteoconductive way to fill voids with a precast cohesive material, for applications such as filling post-extraction tooth sockets or filling other open bone cavities.

In another embodiment, a matt can be cast onto a cement composition of the invention. Alternatively, cement particulates (granules) can be cast within or bonded onto a matt, as described below. Particle density ranges from 0.1 to 5% particle weight to wet collagen fibrillar pellet volume. The matt-cement composites can be used to repair cortical bone defects where the periosteum was removed or destroyed. The cement provides the replacement for the lost bone and the matt provides the replacement for the lost periosteum. The term matt is art recognized and is intended to include those matts described in pending U.S. patent application Ser. No. 09/042,549, entitled “Biopolymer Matt for Use in Tissue Repair and Reconstruction,” filed Mar. 17, 1998, the contents of which are expressly incorporated herein by reference.

As used herein, the term “matt” refers to a biopolymer scaffold comprising a densely packed random array of biopolymer fibrils or bundles of fibrils or particles, e.g., collagen fibrils. Matts which have been dried, as discussed previously, possess a wet tensile strength of at least 0.02 MPa with a preferred strength of greater than 1 MPa and have a collagenase resistance of at least 20 min per mg of collagen at a collagenase concentration of 10 units per 1 cm² of product. Typically the fibrils or bundles of fibrils are between about 0.01 μm and 50 μm in diameter and between about 0.0002 and 5.0 mm in length, preferably 0.1 μm to 20 μm wide and 0.01 mm to 3 mm long. Matts, whether dried or not, possess the following characteristics: (1) physically stable in aqueous solutions; (2) nontoxic to living organisms; (3) can serve as a substrate for cell attachment and growth; (4) approximately 0.01 mm to 20 mm thick, preferably 0.1 to 5.0 mm thick. In a preferred embodiment, the biopolymer matt, matt composite, or matt composition is a collagen matt, collagen matt composite, or collagen matt composition prepared from collagen solution as previously described.

The biopolymers can be used to create matts, matt composites, or matt compositions which can be in any form or shape, e.g., strips, sheets, tubes, etc. In addition, the biopolymers can be used to create matts which can be supported by polymer mesh, e.g., a Teflon® mesh, or used with tissue culture inserts for multiwell plates which can be used as molds in which matt, matt composites, and matt compositions of the invention can be formed on the polycarbonate membrane of the insert. Polymer meshes used with the matt, matt composites, and matt compositions of the invention can expose cells, such as chondrocytes, contained on and within the matt, matt composites, and matt compositions to body tissues and fluids, for example, when the matt, matt composites, and matt compositions are used as support to stimulate formation of bone. Both the meshes and culture inserts have the advantage of providing a means for handling the matt, matt composites, and matt compositions without requiring actual contact with the matt, matt composites, or matt compositions. The forms and shapes in which the matt, matt composites, and matt compositions are made can mimic those of tissues or body parts to be replaced and thus can be used as prostheses or grafts which tissue cells remodel to promote regeneration of a replacement tissue in the recipient.

Selected reinforcing material can be added to the calcium cement or to biopolymer solutions incorporated into the calcium cements of the invention. The reinforcing material should be added to the cement prior to hardening. Such reinforcing materials include biopolymer fibers, threads, e.g., woven or braided threads, and/or fabrics, e.g., non woven fabrics, prepared, for example, by textile methods. Biopolymer threads, e.g., collagen threads, can be prepared by extruding the biopolymer in solution into a coagulation bath and transferring the biopolymer to a bath containing ethanol or acetone or another dehydrating solution. Alternatively, the thread can be dehydrated by subjection to vacuum-drying. The biopolymer thread can then be cross linked by, for example, methods described herein. An example of an apparatus for spinning and processing a biopolymer fiber, e.g., collagen fiber, i described in U.S. Ser. No. 08/333,414, filed Nov. 2, 1994, the contents of which are incorporated herein by references in their entirety. The threads can then be dried, spooled, for example, by pulling the moving thread over more rollers, stretching and drying it and then winding it onto spools. Textile implements can be employed to weave or braid the threads into fabric or other complex forms or constructs for use as described herein.

Biopolymer fabrics, e.g., non woven biopolymer fabrics, are typically composed of a mat of entangled biopolymer fibers of a selected size and density. Typically, the non woven biopolymer fabrics are produced by spooling dry biopolymer fiber onto a drum of circumference equal to that of the length of an individual fiber element. Spooling is continued until the number of wraps of fiber on the drum equals the number of pieces of fiber required for the fabric. A cut is then made across the wound fiber in a direction parallel to the drum axis and the fibers are removed from the drum. The fiber can then be cross linked if it has not been previously cross linked. The fiber is then dispersed in a volume of a buffer solution for a period of time to stabilize its pH and soften the fiber. The fiber is transferred to a volume of water and agitated mechanically to produce entanglement of the fiber strands. The entangled fiber strands are sieved from the water onto a collection screen until they coat the screen in a mat of uniform density. The mat is then dried on the screen or after transfer to another surface, screen, or cell culture device. If desired, the non woven fabric can be cut or punched into smaller shapes after drying.

Macromolecules necessary for cell growth, morphogenesis, differentiation, and tissue building can also be added to the biopolymer molecules or to the biopolymer fibrils or to the cement composition of the invention to further promote cell in growth and tissue development and organization on or within the cement composition or biopolymer construct. The phrase “macromolecules necessary for cell growth, morphogenesis, differentiation, and tissue building” refers to those molecules, e.g., macromolecules such as proteins, which participate in the development of tissue. Such molecules contain biological, physiological, and structural information for development or regeneration of the tissue structure and function. Examples of these macromolecules include, but are not limited to, growth factors, extracellular matrix proteins, proteoglycans, glycosaminoglycans and polysaccharides. Alternatively, the biopolymer matts, matt composites, and matt compositions of the invention can include extracellular matrix macromolecules in particulate form or extracellular matrix molecules deposited by cells or viable cells.

The term “growth factors” is art recognized and is intended to include, but is not limited to, one or more of platelet derived growth factors (PDGF), e.g., PDGF AA, PDGF BB; insulin-like growth factors (IGF), e.g., IGF-I, IGF-II; fibroblast growth factors (FGF), e.g., acidic FGF, basic FGF, β-endothelial cell growth factor, FGF 4, FGF 5, FGF 6, FGF 7, FGF 8, and FGF 9; transforming growth factors (TGF), e.g., TGF-β1, TGF-β1.2, TGF-β2, TGF-β3, TGF-β5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor. Adams et al., “Regulation of Development and Differentiation by the Extracellular Matrix” Development Vol. 117, p. 1183-1198 (1993) (hereinafter “Adams et al.”) and Kreis et al. editors of the book entitled “Guidebook to the Extracellular Matrix and Adhesion Proteins,” Oxford University Press (1993) (hereinafter “Kreis et al.”) describe extracellular matrix components that regulate differentiation and development. Further, Adams et al. disclose examples of association of growth factors with extracellular matrix proteins and that the extracellular matrix is an important part of the micro-environment and, in collaboration with growth factors, plays a central role in regulating differentiation and development. The teachings of Adams et al. and Kreis et al. are incorporated herein by reference. The term encompasses presently unknown growth factors that may be discovered in the future, since their characterization as a growth factor will be readily determinable by persons skilled in the art.

The term “extracellular matrix proteins” is art recognized and is intended to include one or more of fibronectin, laminin, vitronectin, tenascin, entactin, thrombospondin, elastin, gelatin, collagens, fibrillin, merosin, anchorin, chondronectin, link protein, bone sialoprotein, osteocalcin, osteopontin, epinectin, hyaluronectin, undulin, epiligrin, and kalinin. The term encompasses presently unknown extracellular matrix proteins that may be discovered in the future, since their characterization as an extracellular matrix protein will be readily determinable by persons skilled in the art.

The term “proteoglycan” is art recognized and is intended to include one or more of decorin and dermatan sulfate proteoglycans, keratin or keratan sulfate proteoglycans, aggrecan or chondroitin sulfate proteoglycans, heparan sulfate proteoglycans, biglycan, syndecan, perlecan, or serglycin. The term encompasses presently unknown proteoglycans that may be discovered in the future, since their characterization as a proteoglycan will be readily determinable by persons skilled in the art.

The term “glycosaminoglycan” is art recognized and is intended to include one or more of heparan sulfate, chondroitin sulfate, dermatan sulfate, keratan sulfate, hyaluronic acid. The term encompasses presently unknown glycosaminoglycans that may be discovered in the future, since their characterization as a glycosaminoglycan will be readily determinable by persons skilled in the art.

The term “polysaccharide” is art recognized and is intended to include one or more of heparin, dextran sulfate, chitin, alginic acid, pectin, and xylan. The term encompasses presently unknown polysaccharides that may be discovered in the future, since their characterization as a polysaccharide will be readily determinable by persons skilled in the art.

Suitable living cells include, but are not limited to, epithelial cells, e.g., keratinocytes, adipocytes, hepatocytes, neurons, glial cells, astrocytes, podocytes, mammary epithelial cells, islet cells; endothelial cells, e.g., aortic, capillary and vein endothelial cells; and mesenchymal cells, e.g., dermal fibroblasts, mesothelial cells, stem cells, osteoblasts, smooth muscle cells, striated muscle cells, ligament fibroblasts, tendon fibroblasts, chondrocytes, and fibroblasts.

Extracellular matrix particulates or extracellular matrix particulates dispersed or suspended in a vehicle can also be mixed with the calcium cements of the invention and/or supports detailed above, thereby forming a bone precursor composition having extracellular matrix particulates. As used herein, the language “extracellular matrix particulate” refers to a fragment of an extracellular matrix derived from a tissue source formerly having living cells but which has been processed to remove the cells and to retain noncellular extracellular matrix factors such as, for example, growth factors necessary for cell growth, morphogenesis, and differentiation. Methods for forming extracellular matrix particulates for producing graft tissue are disclosed in U.S. patent application Ser. No. 07/926,885, filed Aug. 7, 1992, U.S. patent application Ser. No. 08/302,087, filed Sep. 6, 1994, and U.S. patent application Ser. No. 08/471,535, filed Jun. 6, 1995. The teachings of U.S. patent application Ser. Nos. 07/926,885, 08/302,087, and 08/471,535 (now U.S. Pat. No. 5,800,537) are incorporated herein by reference.

The methods for forming extracellular matrix particulates include freezing a tissue source, e.g., a connective tissue source, having living cells, whereby the living cells are disrupted to form cell remnants consisting of, for example, cytoplasmic and nuclear components. The tissue source is then processed, e.g., by grinding, washing and sieving, to remove the cytoplasmic and nuclear components without removing extracellular matrix including factors necessary for cell growth, migration, differentiation, and morphogenesis. The extracellular matrix is freeze-dried and fragmented, e.g., cryomilled to produce particulates of defined sizes, to produce extracellular matrix particulates.

The extracellular matrix particulates can include extracellular matrix proteins. For example, extracellular matrix particulates obtained from skin include transforming growth factor β1, platelet-derived growth factor, basic fibroblast growth factor, epidermal growth factor, syndecan-1, decorin, fibronectin, collagens, laminin, tenascin, and dermatan sulfate. Extracellular matrix particulates from lung include syndecan-1, fibronectin, laminin, and tenascin. The extracellular matrix particulates can also include cytokines, e.g., growth factors necessary for tissue development. The term “cytokine” includes but is not limited to growth factors, interleukins, interferons and colony stimulating factors. These factors are present in normal tissue at different stages of tissue development, marked by cell division, morphogenesis and differentiation. Among these factors are stimulatory molecules that provide the signals needed for in vivo tissue repair. These cytokines can stimulate conversion of an implant into a functional substitute for the tissue being replaced. This conversion can occur by mobilizing tissue cells from similar contiguous tissues, e.g., from the circulation and from stem cell reservoirs. Cells can attach to the prostheses which are bioabsorbable and can remodel them into replacement tissues.

Extracellular matrix particulates can be obtained from specific tissues. The particulates have two kinds of informational properties. The first is their molecular diversity, and the second is their micro-architecture, both of which are preserved in the preparation of the microparticulates. The preferred associations among the different molecules of the extracellular matrix are also preserved in the preparation of the microparticulates.

The extracellular matrix plays an instructive role, guiding the activity of cells which are surrounded by it or which are organized on it. Since the execution of cell programs for cell division, morphogenesis, differentiation, tissue building and regeneration depend upon signals emanating from the extracellular matrix, three-dimensional scaffolds, such as collagen foams, are enriched with actual matrix constituents, which exhibit the molecular diversity and the microarchitecture of a generic extracellular matrix, and of extracellular matrices from specific tissues.

To provide further cellular and molecular binding sites on the surfaces of the bone precursor compositions and calcium cements to replace, for example, binding sites which have been compromised as a result of the setting process, a coating process can precede or accompany the application of extracellular matrix particulates to these materials. In addition, artificial microstructures, typically having a size in the range of between about 5 and 500 μm, composed of a matrix polymer, such as collagen, combined with other proteins, proteoglycans, glycosaminoglycans, extracellular matrix enzymes, cytokines (including growth factors), and glycosides can be created in the form of wet or dry particulates that can be applied with the coating solution to the surfaces of the bone precursor composition and calcium cement. The selected components can be chemically or electrostatically bound to the bone precursor composition and calcium cement or can be contained in the microparticulate lattice or in a dehydrated form of the lattice. Thus, the invention also pertains to methods for preparing collagen-coated bone precursor compositions and calcium cements and extracellular matrix particulate-coated bone precursor compositions and calcium cements. These methods typically include forming the selected type of bone precursor composition or calcium cement as described herein and applying a collagen solution or an extracellular matrix particulate solution to the bone precursor composition or calcium cement, thereby forming the collagen-coated or extracellular matrix particulate-coated bone precursor composition or calcium cement. The coated bone precursor compositions and calcium cements can be further freeze-dried. In one embodiment, the collagen solution also includes extracellular matrix particulates. Preferably, bone precursor compositions and calcium cements of the present invention include extraceliular matrix particulates in amounts between about 0.05 to about 20 dry weight percent of the compositions.

In one preferred method, the hardened bone precursor composition, in pellet or granular form is contacted with a neutralizing solution such that a neutralized bone precursor composition is prepared. The term “neutralizing solution” is art recognized as intended to include suitable chemical, biochemical, enzymatic or other components which alter the pH of calcium containing materials. For example, neutralizing solutions are selected from CAPS (3-[cyclohexylamino]-1-propanesulfonic acid), triethanolamine, TES(N-tris[hydroxymethyl]methyl-2-aminoethanesulfonic acid), tricine, HEPES (N-2-hydroxyethyl]piperazine-N′-[2-ethanesulfonic acid]) glycine, phosphate buffer solution, bis tris propane, TAPS (N-tris[hydroxymethyl]methyl-3-aminopropane sulfonic acid), AMP (2-amino-2-methyl-1-propanol) and TRIS (tris[hydroxymethyl]aminomethane). A preferred neutralizing solution is tribasic sodium phosphate. Bone precursor materials and calcium cements prepared in this manner can include or can be conditioned with cells described supra.

The bone precursor compositions and calcium cements of the present invention can be used as substrates for cell growth in vitro and in vivo, e.g., for establishing research model systems. For example, in one embodiment, the bone precursor composition calcium cement can be seeded with abnormal cells to study disease states including cancer. In another embodiment, the bone precursor composition or calcium cement and can serve as diagnostic test models for determining chemotherapeutic strategies by selecting for agents capable of killing cancer cells cultivated in or on the cements. In yet another embodiment, the bone precursor compositions or calcium cements can be used to test the toxicity of various substances to which cells in or on the cements are exposed.

The bone precursor composition or calcium cement can also be used as prostheses which can be introduced or grafted into recipients, e.g., such as mammalian recipients, e.g., humans. For example, the bone precursor composition or calcium cement can be used as a prosthesis or to reconstitute, for example, the following types of tissue: connective tissue such as bone or cartilage, and to anchor tissue such as ligament and tendon. Tissue cells seeded into these bone precursor compositions or calcium cements can be obtained from a mammal, e.g., a human. Tissue cells are delivered to the bone precursor composition or calcium cement by first suspending the cells in small volumes of tissue culture medium. The tissue culture medium which contains the cells can then be applied in drops to the bone precursor composition or calcium cement. Alternatively, the bone precursor composition or calcium cement can be placed in a vessel which contains the tissue culture medium and cells in suspension and which shakes such that the tissue culture medium containing the cells is distributed throughout the bone precursor composition or calcium cement. In another embodiment, tissue cells can be suspended in a biopolymer solution e.g., a collagen solution, at low concentrations, at a temperature of about 4° C. to 10° C., and at a pH of about 7.0. The solution containing the cells can then be delivered to the bone precursor composition or calcium cement. As bone precursor composition or calcium cement is warmed to 37° C., the biopolymer solution, e.g., collagen solution, forms a gel on the bone precursor composition or fragmented calcium cement: As-used herein, the term “gel” refers a network or mesh or biopolymer filaments together with an aqueous solution trapped within the network or mesh of biopolymer filaments. An alginate gel for use as a delivery vehicle of cells to the bone precursor composition or fragmented calcium cement of the invention can be produced by addition of calcium which causes polymerization at room temperature and at a neutral pH. Selected epidermal, endodermal, mesenchymal-derived, epithelial, endothelial, or mesothelial cells can then be seeded onto the surface of the gel-coated bone precursor composition or calcium cement.

The bone precursor composition or calcium cement and other forms of biopolymers described herein can be conditioned, e.g., made tissue-ready or established with pre-tissue elements by cells. For example, the bone precursor composition or calcium cement with or without other forms of biopolymers can be seeded with a selected cell type or selected cell types. The cells can then be allowed to grow, proliferate, and secrete factors, e.g., extracellular matrix factors, which attach or adhere to the cement and/or biopolymers that support, for example, cell growth, differentiation, morphogenesis. The cell conditioning of the bone precursor composition or calcium cement and other biopolymer forms described herein serves at least two functions. First, the cells provide chemical conditioning of the bone precursor composition or calcium cement, i.e., the cells secrete extracellular matrix components which attract in growth of cells into the bone precursor composition or calcium cement and biopolymer forms and support the growth and differentiation of the cells in the foams. Second, the cells provide structural conditioning of the bone precursor composition or calcium cement and biopolymer forms, i.e., the cells remodel the bone precursor composition or calcium cement and biopolymer forms to form a scaffold which provides the appropriate physical structure for the type of cells in the tissue which the bone precursor composition or calcium cement is to replace or reconstruct, e.g., the cells arrange themselves in the lacunae. The cell-cement scaffold can be further treated by mechanical and/or electrical conditioning to stimulate further remodeling and strengthening of the material into a bone as cells respond to the applied forces. The bone precursor composition or calcium cement and/or biopolymer forms containing viable cells can be introduced into a recipient subject. Alternatively, the bone precursor composition or calcium cement and/or biopolymer forms containing the cells can be further processed to kill the cells, e.g., freeze-dried to remove antigenic determinants but leave the deposited extracellular matrix macromolecules, and then introduced into a recipient subject.

The invention further includes methods for preparing bone precursor compositions. The methods include combining calcium monopyrophosphate, alpha-calcium sulfate hemihydrate, monobasic calcium phosphate monohydrate and beta-tri calcium phosphate such that bone precursor compositions are prepared. The ingredients for the bone precursor composition can be admixed first as dry components and in the presence of liquid vehicles as described supra to form a paste which can be injected or molded into desired shapes and left to cure, e.g., harden.. In one embodiment, the method includes the step of crushing hardened pellets then sifting and washing the particles to produce the bone precursor composition as granules having a diameter between about 1 to 500 μm, preferably 50 to 500 μm inclusive. If porosity-imparting particles have been included in the cement composition which can then be molded, the hardened cement composition can be treated to dissolve the particles to create the pores prior to implantation, if desired. Alternatively, in vivo dissolution, e.g., bioabsorption, of the particles will create pores within the cement over time.

In yet another embodiment of the present invention, methods for producing or repairing connective tissue in a subject is disclosed. The methods include administering a bone precursor composition to the subject by injection or implantation at the tissue site, wherein the bone precursor composition includes calcium pyrophosphate, calcium phosphate hemihydrate, monobasic calcium phosphate monohydrate and beta-tri calcium phosphate. The language “producing or repairing” is art recognized and is intended to include the ability to cause, enhance, or stimulate tissue to grow or begin growth in a subject.

One advantage of the present invention is that the bone precursor composition includes calcium salts such as calcium pyrophosphate, calcium sulfate hemihydrate, monobasic calcium phosphate monohydrate, and beta-tricalcium phosphate in amounts which help promote the production and/or repair of the connective tissue in the subject. The bone precursor composition preferably has a ratio by weight of monobasic calcium phosphate monohydrate to beta-tri-calcium phosphate of between about 1:2 to about 1:3, preferably about 1:3.5, more preferably about 1:3.75 and most preferably about 1:3.05. The bone precursor composition can be preferably, in the form of granules with a diameter of between about 1 to 500 μm, preferably 50 to 500 μm inclusive. Furthermore, the bone precursor compositions, which can be granulated can include or be conditioned with cells described supra. Alternatively, the bone precursor composition can further include a pharmaceutically acceptable injection vehicle, a biopolymer foam, a therapeutic agent, a biopolymer fiber, acid or pepsin extracted collagen or extracellular matrix particulates.

The bone precursor compositions and calcium cements of the present invention provide advantages over those known in the art. For example, the bone precursor compositions and calcium cements of the invention can be admixed such that setting times between about one to 15 minutes, preferably about 5 to about 10 minutes, can be accomplished, thereby providing the practitioner with sufficient time to formulate the bone precursor bone composition or cement and yet have the material solidify in a relatively short period of time after injection or application to a site in need thereof. Setting times, for example, were estimated by the procedure similar to that used for conventional cements. Set time was considered to be complete the moment a cylindrical rod (stainless steel, 0.18 centimeters diameter, loaded with 60 grams) put vertically on to the specimen no longer left any mark on its surface. Setting time measurements started at the end of the molding operation. The cement continues to cure, e.g., harden, after setting and reaches full compression strength within 48 hours of preparation of the cement. The compressive strength, C, was calculated by dividing the crushing force by the cross-section of a sample, whereas the diametral tensile strength, T, was calculated from the formula T=2F/πLD in which F is the crushing force, L is length and D is diameter. Values for both T and C are expressed in MPa.

Experimental

Preparation of Microfibrillar Collagen:

This procedure produces a semisolid pellet of collagen microfibrils results from centrifugation of a neutralized solution of collagen. Collagen, 0.5 to 15 mg/ml, preferably 3 to 10 mg/ml, pH about 3.0, was neutralized, by mixing with a neutralizing solution. This collagen mixture was treated with either a dilute base or buffer, in a volume ratio of 85-95% collagen to 5-15% buffer. Suitable bases were 0.02 to 2.0 M sodium, anunonium or potassium hydroxide, preferably 0.6 N NaOH at 8.5% volume to 91.5% collagen volume, or buffer, such as 0.02 to 2.0 M, preferably 0.44 M, sodium bicarbonate pH 6 to 14 or 0.02 to 2.0 M, preferably 0.2 M, sodium or potassium phosphate pH 6 to 14, or other buffers useful in broad pH ranges, such as tris or tricine. The collagen then was incubated for at least 30 min at a temperature between 37 and 4° C., preferably 15° C. The pH was fine-tuned to between 5 and 10, preferably pH 6 to 8, using a dilute base, such as 0.02 to 2.0 M sodium, ammonium or potassium hydroxide or additional amounts of the buffers mentioned above with pH's of 10 or above. The neutral collagen formed into fibrils, which after additional incubation time were pelleted by centrifugation between 1000 and 30,000×g. This centrifugation yielded a pellet of 3-100 mg/ml collagen, depending on the starting concentration of the collagen, the total volume and the time spun, usually starting at 5 mg/ml, spinning at 2000×g for 60 min to yield a 10-15 mg/ml pellet (more centrifugation time or a higher speed yielded a higher concentration collagen pellet). The supernatant was discarded, and after gentle stirring to combine the pellet layers, the semisolid pellet was used for the liquid applications listed above (e.g., liquid ingredient mixed with inorganic calcium compounds to make a cement, used to spray onto bleeding wounds to accelerate clotting, or is used as a vehicle to carry particles duringan injection), or was overlaid or combined with cells for cell cultivation or for seeding implant structures for cell conditioning, or was poured into molds or onto hardened cements for freeze drying.

The fibrits or fibril bundles generated, as observed under a light microscope with 1:1 0.5% toluidine blue stain, were between 0.01 μm to 20 μm wide and about 0,01 mm to 3.0 mm long. The collected microfibrillar collagen pellet has a collagen concentration of at least 7 mg/ml and the supernatant collagen concentration is no more than 1 mg/ml. The collected microfibrillar collagen pellet has an absorbance at 410 nm of at least 1.5, preferably over 2.0. The isolated material from the microfibrillar collagen pellet has no low molecular weight collagen degradation products, as can be determined by electrophoretically analyzing denatured and reduced samples on a 10% dodecyl sulfate polyacrylamide gel.

Preparation of Injectable Cements

Criteria: Injectable=mixed paste able to be loaded into a pharmaceutical syringe and injected through a 14 gauge needle. Set time=the time after molding when a 1.5 mm diameter rod weighed down with 60 g no longer leaves an impression on the surface of the cement.

Key: MCPM=monobasic calcium phosphate monohydrate; β-TCP=beta tricalcium phosphate; CSH=(alpha) calcium sulfate hemihydrate; CPP=calcium rophosphate; AFC=acid-extracted collagen microfibrils; PFC=pepsin-extracted collagen microfibrils. The % collagen liquid was calculated as a percent of the dry weight of cement and added to the total, i.e. if 1 g total dry ingredients are used, then 0.33 g liquid collagen was added to mix the cement into a paste for a 33% collagen amount.

| | % | % β- | % | % | % | Type | | Set Time, | Strength, |
|---|---|---|---|---|---|---|---|---|---|
| # | MCPM | TCP | CSH | CPP | Coll | Coll | Injectable | min | MPa |
| 1 | 16 | 64 | 15 | 5 | none | n.a. | no | 17 | 8.6 |
| 2 | 16 | 64 | 15 | 5 | 33 | AFC | no | 14 | 8.6 |
| 3 | 16 | 64 | 15 | 5 | 35 | AFC | yes | 19 | 11.5 |
| 4 | 16 | 64 | 15 | 5 | 35 | PFC | yes | 21 | 7.5 |
| 5 | 20 | 73 | 6 | 1 | 35 | AFC | almost | 11 | 8.3 |
| 6 | 8 | 72 | 15 | 5 | 35 | AFC | no | 18 | 7.1 |
| 7 | 24 | 56 | 15 | 5 | 35 | AFC | yes | 20 | 9.8 |
| 8 | 22 | 67 | 10 | 1 | 35 | AFC | yes | 12 | 10.3 |
| 9 | 22 | 67 | 10 | 1 | 35 | PFC | yes | 10 | 11.3 |

Cement pastes were packed into a mold and were allowed to harden into a uniform geometric shape. Injectability was determined prior to molding and setting me was determined with the cements in the mold. After allowing at least 48 hours to harden, hardened cement pellets from the mold were tested for compressive strength, by taking into account the surface area subjected to compression. Recipe (1) used 33% distilled water to mix the cement into a paste. Recipes 3 and 4, demonstrated that injectability was achieved at the expense of setting time. Recipes 6 and 7 vary the relative concentration of MCPM and β-TCP, with the lower amount being uninjectable and the higher amount being injectable but slowly setting. Recipes 5, 8 and 9 utilize high MCPM while manipulating the concentrations of the CPP and CSH. High MCPM, lower CSH and CPP in 8 and 9 resulted in injectability, quick setting times, and high compressive strength. The effect was noted regardless of extraction method of the collagen (both AFC and PFC can produce this result). The diametral tensile strength of these mixtures was measured at 9 to 10 MPa.

Casting Collagen Foams onto Cements

A mold was constructed with a solid base and wells which consisted of several detachable horizontal layers. The first well layer was assembled onto the bed and a calcium cement was mixed and applied to the wells. The cements were allowed to set for ten minutes and the next layer of the mold was added. Into the next well layer, fibrillar collagen was overlaid on the setting cement surface. The remainder of the mold was assembled and the mold was placed in the freeze dryer. The foam portion of this construct was seeded with chondrocytes for development of an articular cartilage prosthesis. Mechanical conditioning of the construct for articular cartilage, if desired, is achieved by anchoring the chondrocyte-seeded construct in the apparatus described U.S. Pat. No. 5,882,929.

250 μm Cement Particles and Collagen

A. Injectable 250 μm cement particles.

Hardened cement pellets (Recipe #8 described above) were ground in a mortar and pestle. The ground material was sifted to define the size classes. Particular size classes were mixed with microfibrillar collagen and tested for injectability through a 19 ga needle. Particles sifted to a size range of 140 to 250 μm were added in various proportions to acid-extracted fibrillar collagen (AFC). These particles were injectable in the proportion of 1 g particles to 1 g AFC prepared after neutralization with 8% of 0.6 N NaOH. These particles were injectable in the proportion of 1 g particles to 0.75 g AFC prepared after neutralization with 10% 0.2 M dibasic sodium phosphate. These particles were injectable in the proportion of 1 g particles to 0.5 g AFC prepared after neutralization with 10% sodium bicarbonate.

B. Cement particles in single density Foams.

Hardened cement pellets (Recipe #8 described above) are ground in a mortar and pestle. The ground material was sifted to define the size classes. Particles with sizes between 140 μm and 250 μm were collected and 1 g of these particles were placed into a sieve with a pore size of 53 μm. Deionized water was poured in four 25 ml batches onto the particles in the sieve. With swirling of the buffer, fine particles were washed off the larger particles. The particles then were left in the sieve to dry at room temperature. Particles were mixed with pepsin-extracted fibrillar collagen (PFC) in the proportion of 0.5 g particles to 10 ml PFC. The mixtures were dispensed into molds, freeze-dried and UV crosslinked. The resulting foams had an even distribution of particles throughout and after wetting, could support their own weight without disintegrating. The foams then could be implanted to fill in bone cavities or used for tissue culture or implantation.

A. Preparation of Cements including Collagen and Pore-generating Particles

I. Calcium sulfate hemihydrate was mixed into a paste with 36% isotonic saline. Calcium sulfate paste was loaded into pellet molds hardened and dried for two days. Calcium sulfate pellets were crushed and ground in a mortar. Pellet particles were placed in a sieve stack and sifted. Particles with sizes between 140 μm and 400 μm were collected and 2 g of these particles were placed into a sieve with a pore size of 53 μm. A buffer of 10 mM sodium phosphate, pH 7.4 was poured in four 25 ml portions onto the particles in the sieve. The particles and buffer were swirled, causing fine particles to wash through the sieve. The remaining particles were left in the sieve to dry at room temperature.

II. A 22% MCPM:67% β-TCP:10% CSH:1% CPP cement dry mixture was prepared and then washed. Dried 140-400 μm calcium sulfate hemihydrate particles from step I were added to a proportion of 50% dry weight of the mixture. Microfibrillar collagen was then added wet to reach a 35% weight of the final 50:50 mixture. The components were mixed into a paste, loaded into a mold and allowed to set, harden and dry. Compression tests at 48 h demonstrated a 3.3 MPa strength of the 50:50 pellets.

The product of the 22:67:10:1 hardening reaction did not dissolve readily in deionized water; pellets were placed in deionized water to dissolve the calcium sulfate particles and produce pores in the pellets, when could then be implanted into bone voids. Alternatively, instead of pre-dissolving the calcium sulfate, the pellets could be implanted to fill bone voids and allow biological processes to dissolve the calcium sulfate particles before dissolving the pellet superstructure to allow pore formation to occur gradually during the process of bone ingrowth.

B. Preparation of Cements including Collagen and Pore-generating Particles

The above experiment was conducted generally as above, with changes and observations in the above procedure noted as follows. In Step I, calcium sulfate paste was loaded into pellet molds and allowed to harden and dry for two days. Calcium sulfate pellets were then crushed and ground in a mortar and pestle, and pellet particles were placed in a sieve stack and sifted. Particles with sizes between 250 μm and 400 μm were collected, and 2 g of these particles were placed into a sieve with a pore size of 53 μm. With swirling of the sodium phosphate buffer added to the sieve as above and draining through the sieve, fine particles were washed through the sieve off the larger particles which remained in the sieve. The particles then were left in the sieve to dry at room temperature.

Step II. A 22% MCPM:67% β-TCP:10% CSH:1% CPP cement dry mixture was prepared as above, but washed, dried 250-400 μm calcium sulfate hemihydrate particles from step I were added to a proportion of 30 % dry weight. Microfibrillar collagen then was added wet to reach a 40% weight of the final 70:30 mixture. The components were mixed into a paste, which was confirmed to be injectable, loaded into a mold and allowed to set (a 9 min set time was measured), harden and dry. Compression tests at 48 h demonstrated a 16 MPa strength of the 70:30 pellets compared to 22 MPa of pellets without particles. The standard product of the 22:67:10:1 hardening reaction does not dissolve readily in deionized water. Therefore, the pellets with the calcium sulfate particles were placed in deionized water to dissolve the calcium sulfate particles and produce pores in the pellets, which then could be implanted into bone voids. Particle-containing pellets placed in deionized water for one day exhibit pores up to 250 μm diameter and absorb liquid at a rate 4 times faster than before creating the pores. More pores form with additional time in deionized water, so after two days, the liquid absorption rate is 14 times faster than before creating the pores. Alternatively, instead of pre-dissolving the calcium sulfate, the pellets could be implanted to fill bone voids and allow biological processes to dissolve the calcium sulfate particles before dissolving the pellet superstructure to allow pore formation gradually during the process of bone ingrowth.

Cultivation of mammalian Cells on Cement constructs

The following demonstrative cultivation of cells on cement pellets, on cement microparticulates or in foams containing cement microparticulates. The cement ingredients were sterilized by methods standard to the art. Cements were aseptically measured, mixed, molded into pellets and allowed to harden. If microparticulates were used, then the pellets were ground and sifted to desired size classes. If particulates were embedded in foams, the method of example B was followed. For small volume culture, cements first conducted buffer conditioning. For example, 2.4 g of cement pellets (Recipe #8 described above) was washed for 6 hours in 30 mL 0.05 M tribasic sodium phosphate, pH 12, followed by water and phosphate-buffered saline rinses prior to equilibrating in culture medium. For larger volume cultures, rinses and soaks in culture medium were sufficient to ensure pH compatibility of cement constructs with cell culture (the pH of hardened cements is ˜5.5, low for tissue culture). Suspensions of 1 to 5×10⁵ cells per ml were added to cements for seeding 1 to 2 h while gently agitating. After seeding, excess unattached cells were removed with a change of culture medium and cement constructs were returned to the incubator for further incubation with the loose cement particulates continuously being gently agitated. Cells were cultivated on cements or particulates for as long as desired, given sufficient medium changes. Cells were observed during culture after staining with fluorescent dye. Metabolic assays were performed with the cells on the cements or after cells were released from cements by trypsin. Cements or particulates with cells were fixed and prepared for histology and immunohistochemical staining.

Cement constructs supporting cell growth for sufficient time, 7 days to 3 weeks, for the cells to deposit extracellular matrix on the cements were treated further as cell-conditioned cements. For this process, if desired, the cement construct were treated by mild solutions to lyse cells and release intracellular contents. Regardless of whether cell washing was undertaken, the construct was washed in dilute neutral buffer and freeze-dried. These freeze-dried materials were used as cell-conditioned products with much of the tissue foundation already deposited on the cement construct and implanted by methods appropriate for each construct format for rapid tissue induction.

Equivalents

The features and other details of the invention will now be more particularly described and pointed out in the claims. It will be understood that the particular embodiments of the invention are shown by way of illustration and not as limitations of the of the invention. The principal features of this invention can be employed in various embodiments without departing from the scope of the invention.

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A bone precursor composition, comprising
a calcium cement which is suitable for injection, wherein the calcium cement includes monobasic calcium phosphate monohydrate and beta-tricalcium phosphate.

2. The composition of claim 1, further comprising calcium pyrophosphate and alpha-calcium sulfate hemihydrate.

3. The composition of claim 2, wherein the ratio by weight of monobasic calcium phosphate monohydrate to beta-tricalcium phosphate is 1:2 to 1:3.75.

4. The composition of claim 1, wherein the calcium cement is in the form of granules with a diameter of between about 1 to 500 μm inclusive.

5. The composition of claim 4, which includes or is conditioned with cells.

6. The composition of claim 5, wherein the cells are tissue cells or mesenchymal cells.

7. The composition of claim 6, wherein the mesenchymal cells are connective tissue cells or bone cells.

8. The composition of claim 7, wherein the connective tissue cells are selected from the group consisting of ligament cells and chondrocytes and tendon cells.

9. The composition of claim 7, wherein the bone cells are selected from the group consisting of bone marrow stem cells, osteocytes, osteoblasts and osteoclasts.

10. The composition of claim 1, further comprising an injection vehicle.

11. The composition of claim 10, wherein the injection vehicle is selected from the group consisting of microfibrillar collagen and unassembled liquid collagen.

12. The composition of claim 11, wherein said injection vehicle is unassembled liquid collagen in a concentration from about 0.5 mg/ml to about 40 mg/ml.

13. The composition of claim 10, wherein said injection vehicle further comprises collagen foam, collagen fiber particles, methyl cellulose, or a pharmaceutically acceptable vehicle.

14. The composition of claim 10, wherein said calcium cement comprises calcium pyrophosphate, alpha calcium sulfate hemihydrate, monobasic calcium phosphate monohydrate and beta tricalcium phosphate.

15. The composition of claim 2, wherein said calcium cement comprises, by weight, between about 1 and 5 percent calcium pyrophosphate, between about 5 and 15 percent alpha-calcium sulfate hemihydrate, between about 5 and 25 percent monobasic calcium phosphate monohydrate and between about 55 and 75 percent beta-tricalcium phosphate.

16. The composition of claim 1, further comprising a therapeutic or analgesic agent.

17. The composition of claim 11, wherein the collagen is fetal porcine collagen.

18. The composition of claim 1, further comprising macromolecules necessary for cell growth, morphogenesis, differentiation and tissue building.

19. The composition of claim 18, wherein the macromolecules are in the form of extracellular matrix particulates.

20. The composition of claim 19, wherein the extracellular matrix particulates comprise between about 0.05 to 20 weight percent of the composition when dry.

21. The composition of claim 1, further comprising pore-generating particles.

22. The composition of claim 21, wherein said pore-generating particles are selected from the group consisting of gelatin and calcium sulfate, or mixtures thereof

23. A bone precursor composite, comprising
a calcium cement; and
a biopolymer structure.

24. The composite of claim 23, wherein said biopolymer structure is collagen.

25. The composite of claim 24, wherein the collagen is fetal porcine collagen.

26. The composite of claim 23 wherein the biopolymer structure is a sponge or a single density foam.

27. The composite of claim 23 wherein the biopolymer structure is a fiber or fibers.

28. The composite of claim 23 wherein the biopolymer structure is a matt.

29. The composite of claim 23 wherein the biopolymer structure is a double density foam.

30. The composite of claim 23 wherein the biopolymer structure is a composite of a biopolymer structure and another structure.

31. The composite of claim 23, wherein the biopolymer foam and/or the calcium cement includes or is conditioned with cells.

32. The composite of claim 31, wherein said composition is mechanically conditioned.

33. A bone precursor composition, comprising
a calcium cement; and
acid or pepsin extracted collagen.

34. The composition of claim 33, wherein the collagen is in the form of lyophilized collagen.

35. The composition of claim 33, wherein the collagen is microfibrillar collagen.

36. The composition of claim 33, wherein the calcium cement includes calcium salts selected from the group consisting of calcium pyrophosphate, alpha-calcium sulfate hemihydrate, monobasic calcium phosphate monohydrate, beta-tricalcium phosphate, and mixtures thereof.

37. The composition of claim 34, wherein the collagen comprises between about 0.1 to 2.5 weight percent of the composition when dry.

38. The composition of claim 36, wherein the ratio by weight of monobasic calcium phosphate monohydrate to beta-tricalcium phosphate is 1:2.5 to 1:3.75.

39. The composition of claim 33, wherein the calcium cement is in the form of granules with a diameter of between about 1 to 500 μm inclusive.

40. A method for preparing an injectable bone precursor composition, comprising combining calcium pyrophosphate, alpha-calcium sulfate hemihydrate, monobasic calcium phosphate monohydrate and beta-tricalcium phosphate, such that an injectable bone precursor composition is prepared.

41. The method of claim 40, wherein the ratio by weight of monobasic calcium phosphate monohydrate to beta-tricalcium phosphate is 1:2.5 to 1:3.75.

42. The method of claim 40, further comprising the step of producing the bone precursor composition as granules of reacted, hardened cement having a diameter of between about 1 to 500 μm inclusive.

43. The method of claim 40, further comprising the step of contacting the bone precursor composition with a neutralizing solution such that a neutralized bone precursor composition is prepared.

44. The method of claim 43, wherein the neutralizing solution is selected from the group consisting of CAPS, triethanolamine, TES, tricine, HEPES, glycine, phosphate buffer solution, bis tris propane, TAPS, AMP and TRIS.

45. The method of claim 43, wherein the neutralizing solution is tribasic sodium phosphate.

46. A method for producing or repairing connective tissue in a subject, comprising administering an injectable bone precursor composition to the subject, wherein the injectable bone precursor composition comprises calcium pyrophosphate, calcium sulfate hemihydrate, monobasic calcium phosphate monohydrate and beta-tricalcium phosphate.

47. The method of claim 46, wherein the ratio by weight of monobasic calcium phosphate monohydrate to beta-tricalcium phosphate is 1:2 to 1:3.75.

48. The method of claim 46, wherein the bone precursor composition is in the form of granules with a diameter of between about 1 to 500 μm inclusive.

49. The method of claim 46, wherein the bone precursor composition includes or is conditioned with cells.

50. The method of claim 46, wherein the cells are tissue cells or mesenchymal cells.

51. The method of claim 46, wherein the bone precursor composition further comprises an injection vehicle.

52. The method of claim 46, wherein the bone precursor composition further comprises a biopolymer structure.

53. The method of claim 46, wherein the bone precursor composition further comprises a therapeutic and/or analgesic agent.

54. The method of claim 46, wherein the bone precursor composition further comprises acid or pepsin extracted collagen.

55. The method of claim 46, wherein the bone precursor composition further comprises extracellular matrix particulates.

56. The method of claim 46, wherein the bone precursor composition further comprises pore-generating particles.
